(19) United States
(12) Patent Application Publication
(10) Pub. No.: US 2008/0035141 A1
(43) Pub. Date: Feb. 14, 2008

(54) AEROSOLIZED THERAPY KIT

(76) Inventors: W. Randolph Warner, Punta Gorda, FL (US); Werner Gutmann, Powhatan, VA (US)

Correspondence Address: MCGUIREWOODS, LLP, 1750 TYSONS BLVD, SUITE 1800, MCLEAN, VA, 22102, US

(21) Appl. No.: 11/763,975

(22) Filed: Jun. 15, 2007

Related U.S. Application Data
(60) Provisional application No. 60/814,060, filed on Jun. 16, 2006.

(51) Int. Cl. A61M 11/00; A61K 31/56; A61K 38/02; A61P 43/00; A61P 11/00; A61K 38/16

(52) U.S. Cl. 128/200.14; 514/12; 514/169; 514/2; 514/789

(57) ABSTRACT

A comprehensive aerosol therapy for administration to a patient in need of treatment is provided. More particularly, the aerosol therapy kit provides a seamless conduit for a patient to receive in-office or hospital inhalation treatments and access to drugs and equipment for continuing home use.

### CROSS-REFERENCE TO RELATED APPLICATIONS

This application claims priority under 35 U.S.C. §119(e) to provisional U.S. Patent Application No. 60/814,060, filed on Jun. 16, 2006; the disclosure of which are expressly incorporated by reference herein in its entirety.

### FIELD OF THE INVENTION

The invention relates to a comprehensive unit to administer aerosol therapy to a patient in need of treatment. More particularly, the invention relates to an aerosol therapy kit to provide a seamless conduit for a patient to receive in-office or hospital inhalation treatments and access to drugs and equipment for continuing home use.

### BACKGROUND OF THE INVENTION

There are a number of types of medical procedure kits. Medical procedure kits may be used for many medical and/or surgical procedures such as laparoscopic or endoscopic surgery. The kits are provided with several components used in connection with the specific medical procedure to be performed. These kits have been used for example, as suture or wound closure kits which include the tools necessary to complete a desired medical procedure.

A surgical suture kit, for example, may include a disposable suture passer, a pilot suturing guide, and braided sutures. Such a kit may be intended for use in securing trocar wounds made during laparoscopic surgery. Additionally, a procedure kit useful in performing laparoscopic or endoscopic surgery may include instrumentation such as a trocar assembly, an obturator, a sleeve member, a cutting device, a stapling device, a dissector, a gripping device, and a catheter.

Hospital emergency rooms, hospital emergency departments, and in-offices may treat many out-patients with aerosolized drug treatments. However, each caregiver must source the items needed such as nebulizers, tubing, masks, compressors (offices and ambulatory clinics) and drugs. In most cases, these items are outsourced from various vendors, distributors and manufacturers. Thus, it would be desirable to provide a package and/or kit for a nebulizer in combination with a number of other components necessary for a caregiver to administer aerosolized therapy to a patient. The other items in the kit may include tubing, masks, compressors and drugs.

### SUMMARY OF THE INVENTION

The invention satisfies the above needs by providing a comprehensive kit for a caregiver to administer aerosol therapy to a patient in need.

In one aspect of the invention an aerosol therapy kit is provided. The kit may include a nebulizer, at least one vial containing an effective amount of at least one drug, and at least one pre-printed prescription. Moreover, the kit may also include nebulizer tubing and a mask.

In a further aspect of the invention, the drug in the kit may include a compound selected from the group consisting of anti-inflammatory compounds, anti-allergies, glucocorticoids, anti-infective agents, antibiotics, antifungals, antivirals, mucolytics, antiseptics, vasoconstrictors, wound healing agents, local anesthetics, peptides, and proteins. Moreover, the effective amount of the drug is used to treat a patient afflicted with a condition selected from the group consisting of asthma, acute lower respiratory tract infection, chronic obstructive pulmonary disease, acute bronchitis, bronchiectasis, environmental pulmonary diseases, pulmonary hypertension, mediastinal and pleural disorders, pneumonia, infant respiratory distress syndrome, croup, bronchitis, and pertussis

In another aspect, the nebulizer may be suitable for use with an adult, child or an infant. Furthermore, the nebulizer may be a jet nebulizer or a mesh nebulizer.

### BRIEF DESCRIPTION OF THE DRAWINGS

The accompanying drawings, which are included to provide a further understanding of the invention, are incorporated in and constitute a part of this specification, illustrate embodiments of the invention and together with the detailed description serve to explain the principles of the invention. No attempt is made to show structural details of the invention in more detail than may be necessary for a fundamental understanding of the invention and various ways in which it may be practiced.

FIG. 1 shows an aerosol therapy kit of the invention, according to principles of the invention.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. This, for example, a reference to “a construct” is a reference to one or more constructs and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Moreover, provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

### DEFINITIONS

The terms “active agent,” “drug” and “pharmacologically active agent” are used interchangeably herein to refer to a chemical material or compound which, when administered to an organism (human or animal) induces a desired pharmacologic effect. Included are derivatives and analogs of those compounds or classes of compounds specifically mentioned that also induce the desired pharmacologic effect.

By the terms “effective amount” or “therapeutically effective amount” of an agent as provided herein are meant a nontoxic but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using only routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” asthma, as the term “treating” is used herein, encompasses both prevention of asthma in a predisposed individual and treatment of asthma in a clinically symptomatic individual.

The terms “condition,” “disease” and “disorder” are used interchangeably herein as referring to a physiological state that can be prevented or treated by administration of a pharmaceutical formulation as described herein. Exemplary diseases and conditions may include, but not limited to, asthma, acute lower respiratory tract infection, chronic obstructive pulmonary disease, acute bronchitis, bronchiectasis, environmental pulmonary diseases, pulmonary hypertension, mediastinal and pleural disorders, pneumonia, infant respiratory distress syndrome, croup, bronchitis, and pertussis.

The term “patient” as in treatment of “a patient” refers to a mammalian individual afflicted with or prone to a condition, disease or disorder as specified herein, and includes both humans and animals.

An aerosol, as used herein, is a system comprising a continuous gas phase and, dispersed therein, a discontinuous phase of liquid and/or solid particles.

Nebulization, as used herein, refers to the conversion of a liquid, such as a liquid solution, emulsion, or suspension, into an aerosol. Thus, a nebulized aerosol comprises liquid droplets dispersed in a continuous gas phase. The liquid droplet may optionally comprise solid particles which are suspended within the droplets.

A nebulizer, as used herein, is a device which is capable of converting a liquid material into a nebulized aerosol which is typically inhalable by a human via the nose or the mouth.

The invention relates to a therapy kit to provide healthcare givers a comprehensive unit to provide aerosolized drug treatments to a patient. More particularly, the aerosolized therapy kits of the invention may be comprehensive kits for the treatment of pulmonary diseases or pulmonary infection, such as adult or childhood asthma or croup.

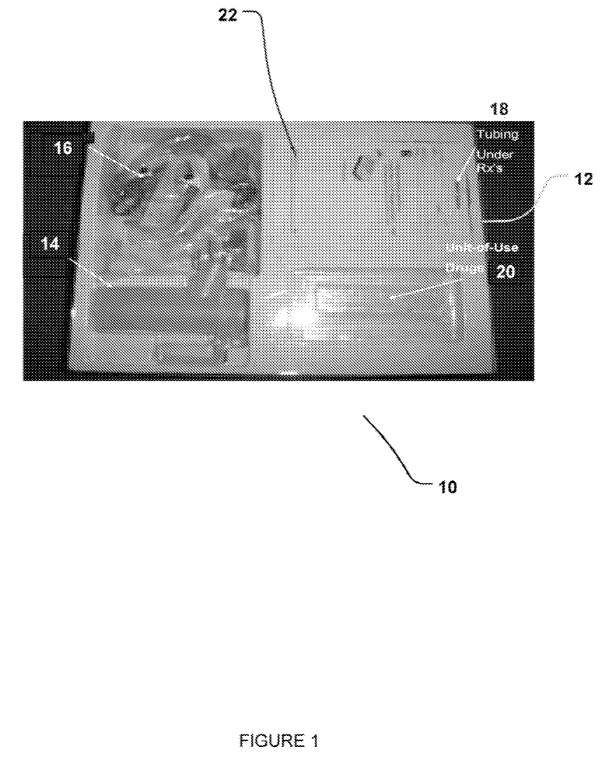

The aerosol therapy kit may contain all items that are needed for a caregiver to administer aerosol therapy to a patient. For example, in general, the aerosol therapy kit may contain at least one nebulizer, at least one drug to be administered to the patient, and at least one pre-filled prescription form for follow-up or preventive treatment. Additionally, the aerosol therapy kit may further include at least one mask and nebulizer tubing. FIG. 1, which shows an embodiment of the invention, shows an aerosol therapy kit 10 containing a thermoform tray 12, a nebulizer 14, a nebulizer mask 16, tubing 18, unit-of-use drug 20, and pre-filled or pre-printed prescriptions 22.

The contents of each kit would depend upon the type of treatment to be administered to a patient. According to one embodiment, the aerosol therapy kit may be used for the treatment of asthma. Here, the aerosol kit of the invention may contain a nebulizer, tubing, a pediatric mask, a 3 ml vial of the therapeutic drug, such as albuterol, xopenex or leval buteral, a 5 ml unit-dose vial of liquid oral prednisolone soldium phosphate (15 mg/ml) and 1 respule of pulmicort and/or budesnoide, and pre-printed prescriptions for albuterol, xopenex, leavalbuterol, PARI pred-pack (pulmocort, budesnoide). In a further embodiment, the aerosol kit may also contain a nebulizer compressor system.

According to another embodiment, the aerosol therapy kit may be used for the treatment of croup. Here, the aerosol kit of the invention may contain a nebulizer, tubing, a mask appropriate for use with an infant, 3 ml vial of racemic epinephrine, a 5 ml unit-dose vial of liquid oral predniosolone sodium phosphate (15 mg/ml), and pre-printed prescriptions for PARI Pred-Pack.

There are two widely known classes of medical nebulizers: the jet nebulizer, which is powered by compressed air, and the ultrasonic nebulizer, which derives the energy required to aerosolize drugs from high frequency sound waves. Jet nebulizers are driven either by a portable compressor or from a central air supply. Among jet nebulizers which meet the criteria for carrying out the invention include, for example, high-performance devices such as the PARI LC or PARI LC SPRINT, driven by an appropriate compressor such as the PARI Boy N (=PRONEB ultra in the USA). In particular, a nebulizer may be used which also achieves a very high FPF such as an electronic perforated vibrating membrane nebulizer, and may include a member of the PARI eFlow series. Other optional nebulizers may include ultrasonic nebulizers, electrohydrodynamic nebulizers, non-vibrating or nonperforated membrane nebulizers, or nebulizers combining two or more of these types.

The aerosol therapy kits of the invention may be assembled or modified to be suitable for treatment of any pulmonary disorders and diseases. For example, the aerosol therapy kits may be used for the treatment of asthma, acute lower respiratory tract infection, chronic obstructive pulmonary disease, acute bronchitis, bronchiectasis, environmental pulmonary diseases, pulmonary hypertension, mediastinal and pleural disorders, pneumonia, infant respiratory distress syndrome, croup, bronchitis, and pertussis. For example, an aerosol therapy kit may be assembled which includes an effective amount of a drug, such as short-acting bronchodilator/beta agonist pulmicort respules, budesonide inhalation suspension, cortico-steroid/anti-inflammatory racepinephrine, and (racemic epinephrine) bronchodilator.

Further exemplary drug compounds for use in the aerosol therapy kit of the invention, may include but not limited to, alprazolam; amoxapine; atropine; bumetanide; buprenorphine; butorphanol; clomipramine; donepezil; hydromorphone; loxapine; midazolam; morphine; nalbuphine; naratriptan; olanzapine; paroxetine; pramipexole; prochlorperazine; quetiapine; rizatriptan; sertraline; sibutramine; sildenafil; sumatriptan; tadalafil; vardenafil; venlafaxine; zolpidem; apomorphine HCl; celecoxib; ciclesonide; eletriptan; parecoxib; valdecoxib; fentanyl; citalopram; escitalopram; clonazepam; oxymorphone; albuterol; sufentanyl; and remifentanyl.

Additionally, the drug vials provided in the therapy kit may contain an effective dose of, for example, substances selected from the groups of anti-inflammatory compounds, anti-allergies, glucocorticoids, anti-infective agents, antibiotics, antifungals, antivirals, mucolytics, antiseptics, vasoconstrictors, wound healing agents, local anesthetics, peptides, and proteins.

Examples of potentially useful anti-inflammatory compounds that may be applicable for aerosol therapy may include glucocorticoids and non-steroidal anti-inflammatory agents such as betamethasone, beclomethasone, budesonide, ciclesonide, dexamethasone, desoxymethasone, fluoconolone acetonide, flucinonide, flunisolide, fluticasone, icomethasone, rofleponide, triamcinolone acetonide, fluocortin butyl, hydrocortisone, hydroxycortisone-17-butyrate, prednicarbate, 6-methylprednisolone aceponate, mometasone furoate, elastane, prostaglandin, leukotriene, bradykinin antagonists, non-steroidal anti-inflammatory drugs (NSAIDs), such as ibuprofen, indometacin, including any pharmaceutically acceptable salts, esters, isomers, stereoisomers, diastereomers, epimers, solvates or other hydrates, prodrugs, derivatives, or any other chemical or physical forms of active compounds comprising the respective active moieties.

Examples of potentially useful antiallergic agents applicable for aerosol therapy may include glucocorticoids, nedocromil, cetrizin, loratidin, montelukast, roflumilast, ziluton, omalizumab, Heparinoids and other antihistamins, Azelastine, Cetirizin, Desloratadin, Ebastin, Fexofenadin, Levocetirizin, Loratadin.

Examples of anti-infective agents, whose class or therapeutic category is herein understood as comprising compounds which are effective against bacterial, fungal, and viral infections, i.e. encompassing the classes of antimicrobials, antibiotics, antifungals, antiseptics, and antivirals, that may be suitable for aerosol therapy may include penicillins, including benzylpenicillins (penicillin-G-sodium, clemizone penicillin, benzathine penicillin G), phenoxypenicillins (penicillin V, propicillin), aminobenzylpenicillins (ampicillin, amoxycillin, bacampicillin), acylaminopenicillins (azlocillin, mezlocillin, piperacillin, apalcillin), carboxypenicillins (carbenicillin, ticarcillin, temocillin), isoxazolyl penicillins (oxacillin, cloxacillin, dicloxacillin, flucloxacillin), and amidine penicillins (mecillinam); cephalosporins, including cefazolins (cefazolin, cefazedone); cefuroximes (cerufoxim, cefamdole, cefotiam), cefoxitins (cefoxitin, cefotetan, latamoxef, flomoxef), cefotaximes (cefotaxime, ceftriaxone, ceftizoxime, cefmenoxime), ceftazidimes (ceftazidime, cefpirome, cefepime), cefalexins (cefalexin, cefaclor, cefadroxil, cefradine, loracarbef, cefprozil), and cefiximes (cefixime, cefpodoxim proxetile, cefuroxime axetil, cefetamet pivoxil, cefotiam hexetil), loracarbef, cefepim, clavulanic acid/amoxicillin, Ceftobiprole; synergists, including beta-lactamase inhibitors, such as clavulanic acid, sulbactam, and tazobactam; carbapenems, including imipenem, cilastin, meropenem, doripenem, tebipenem, ertapenem, ritipenam, and biapenem; monobactams, including aztreonam; aminoglycosides, such as apramycin, gentamicin, amikacin, isepamicin, arbekacin, tobramycin, netilmicin, spectinomycin, streptomycin, capreomycin, neomycin, paromoycin, and kanamycin; macrolides, including erythromycin, clarythromycin, roxithromycin, azithromycin, dithromycin, josamycin, spiramycin and telithromycin; gyrase inhibitors or fluoroquinolones, including ciprofloxacin, gatifloxacin, norfloxacin, ofloxacin, levofloxacin, perfloxacin, lomefloxacin, fleroxacin, garenoxacin, clinafloxacin, sitafloxacin, prulifloxacin, olamufloxacin, caderofloxacin, gemifloxacin, balofloxacin, trovafloxacin, and moxifloxacin; tetracyclins, including tetracyclin, oxytetracyclin, rolitetracyclin, minocyclin, doxycycline, tigecycline and aminocycline; glycopeptides, including vancomycin, teicoplanin, ristocetin, avoparcin, oritavancin, ramoplanin, and peptide 4; polypeptides, including plectasin, dalbavancin, daptomycin, oritavancin, ramoplanin, dalbavancin, telavancin, bacitracin, tyrothricin, neomycin, kanamycin, mupirocin, paromomycin, polymyxin B and colistin; sulfonamides, including sulfadiazine, sulfamethoxazole, sulfalene, co-trimoxazole, co-trimetrol, co-trimoxazine, and co-tetraxazine; azoles, including clotrimazole, oxiconazole, miconazole, ketoconazole, itraconazole, fluconazole, metronidazole, timidazole, bifonazol, ravuconazol, posaconazol, voriconazole, and omidazole and other antifungals including flucytosin, griseofluvin, tonoftal, naftifin, terbinafin, amorolfin, ciclopiroxolamin, echinocandins, such as micafungin, caspofungin, anidulafungin; nitrofurans, including nitrofurantoin and nitrofuranzone; -polyenes, including amphotericin B, natamycin, nystatin, flucocytosine; other antibiotics, including tithromycin, lincomycin, clindamycin, oxazolindiones (linzezolids), ranbezolid, streptogramine A+B, pristinamycin aA+B, Virginiamycin A+B, dalfopristin/qiunupristin (Synercid), chloramphenicol, ethambutol, pyrazinamid, terizidon, dapson, prothionamid, fosfomycin, fucidinic acid, rifampicin, isoniazid, cycloserine, terizidone, ansamycin, lysostaphin, iclaprim, mirocin B17, clerocidin, filgrastim, and pentamidine; antivirals, including aciclovir, ganciclovir, birivudin, valaciclovir, zidovudine, didanosin, thiacytidin, stavudin, lamivudin, zalcitabin, ribavirin, nevirapirin, delaviridin, trifluridin, ritonavir, saquinavir, indinavir, foscarnet, amantadin, podophyllotoxin, vidarabine, tromantadine, and proteinase inhibitors; plant extracts or ingredients, such as plant extracts from chamomile, hamamelis, echinacea, calendula, papain, pelargonium, essential oils, myrtol, pinen, limonen, cineole, thymol, mentol, alpha-hederin, bisabolol, lycopodin, vitapherole; wound healing compounds including dexpantenol, allantoin, vitamins, hyaluronic acid, alpha-antitrypsin, anorganic and organic zinc salts/compounds, interferones (alpha, beta, gamma), tumor necrosis factors, cytokines, interleukins.

Examples of potentially useful mucolytics that may be useful for aerosol therapy may be DNase, P2Y2-agonists (denufosol), heparinoids, guaifenesin, acetylcysteine, carbocysteine, ambroxol, bromhexine, lecithins, myrtol, and recombinant surfactant proteins.

Examples of potentially useful local anaesthetic agent which may be suitable for aersol therapy may include benzocaine, tetracaine, procaine, lidocaine and bupivacaine.

Examples of potentially useful antiallergic agents which may be applicable for aerosol therapy may include the glucocorticoids, cromolyn sodium, nedocromil, Examples of potentially useful peptides and proteins include antibodies against toxins produced by microorganisms, antimicrobial peptides such as cecropins, defensins, thionins, and cathelicidins.

Additionally drugs to treat pulmonary hypertension, such as prostacycline analogs, iloprost, remodulin, phosphodiesterase inhibitors, such as sildenafil, vardenafil, endothelian recector antagonists, such as bosentane, virustatics, including podophyllotoxine, vidarabine, tromantadine, zidovudine; ribavirin, may be applicable for aerosol therapy.

Also, immunmodulators may be suitable for aerosol therapy may include methotrexat, azathioprine, cyclosporine, tacrolimus, sirolimus, rapamycin, mofetil, cytotatics and metastasis inhibitors, alkylants, such as nimustine, melphanlane, carmustine, lomustine, cyclophosphosphamide, ifosfamide, trofosfamide, chlorambucil, busulfane, treosulfane, prednimustine, thiotepa; antimetabolites, e.g. cytarabine, fluorouracil, methotrexate, mercaptopurine, tioguanine; alkaloids, such as vinblastine, vincristine, vindesine; antibiotics, such as alcarubicine, bleomycine, dactinomycine, daunorubicine, doxorubicine, epirubicine, idarubicine, mitomycine, plicamycine; complexes of secondary group elements (e.g. Ti, Zr, V, Nb, Ta, Mo, W, Pt) such as carboplatinum, cis-platinum and metallocene compounds such as titanocendichloride; amsacrine, dacarbazine, estramustine, etoposide, beraprost, hydroxycarbamide, mitoxanthrone, procarbazine, temiposide; paclitaxel, iressa, zactima, poly-ADP-ribose-polymerase (PRAP) enzyme inhibitors, banoxantrone, gemcitabine, pemetrexed, bevacizumab, ranibizumab.

In a further embodiment other active ingredient that may used in aerosol therapy may include, proteinase inhibitors, such as a-anti-trypsin; antioxidants, such as tocopherols, glutathion; pituitary hormones, hypothalamic hormones, regulatory peptides and their inhibiting agents, corticotropine, tetracosactide, choriogonandotropine, urofolitropine, urogonadotropine, saomatotropine, metergoline, desmopressine, oxytocine, argipressine, ornipressine, leuproreline, triptoreline, gonadoreline, busereline, nafareline, goselerine, somatostatine; parathyroide gland hormones, calcium metabolism regulators, dihydrotachysterole, calcitonine, clodronic acid, etidronic acid; thyroid gland therapeutics; sex hormones and their inhibiting agents, anabolics, androgens, estrogens, gestagenes, antiestrogenes; anti-migraine drugs, such as proxibarbal, lisuride, methysergide, dihydroergotamine, ergotamine, clonidine, pizotifene; hypnotics, sedatives, benzodiazepines, barbiturates, cyclopyrrolones, imidazopyridines, antiepileptics, zolpidem, barbiturates, phenyloin, primidone, mesuximide, ethosuximide, sultiam, carbamazepin, valproic acid, vigabatrine; antiparkinson drugs, such as levodopa, carbidopa, benserazide, selegiline, bromocriptine, amantadine, tiapride; antiemetics, such as thiethylperazine, bromopride, domperidone, granisetrone, ondasetrone, tropisetrone, pyridoxine; analgesics, such as buprenorphine, fentanyl, morphine, codeine, hydromorphone, methadone, fenpipramide, fentanyl, piritramide, pentazocine, buprenorphine, nalbuphine, tilidine; drugs for narcosis, such as N-methylated barbiturates, thiobarbiturates, ketamine, etomidate, propofol, benzodiazepines, droperidol, haloperidol, alfentanyl, sulfentanyl; antirheumatism drugs including tumor necrosis factor-alfa, nonsteroidal antiinflammatory drugs; antidiabetic drugs, such as insulin, sulfonylurea derivatives, biguanids, glitizols, glucagon, diazoxid; cytokines, such as interleukines, interferones, tumor necrosis factor (TNF), colony stimulating factors (GM-CSF, G-CSF, M-CSF); proteins, e.g. epoetine, and peptides, e.g. parathyrin, somatomedin C; heparine, heparinoids, urokinases, streptokinases, ATP-ase, prostacycline, sexual stimulants, or genetic material.

Additional constituent elements of the formulation of the invention may include water, a buffer, a pH-adjusting agent, a surfactant or anti-adsorbent, a wetting agent, a gelling agent, a drying agent, an osmolality adjusting agent, or virtually any other additive or carrier, depending upon the desired dosage form.

Additionally, polymeric excipients may be useful in the formulations of the invention, for among other things, to obtain slow release profile of the drug, such as chitosan,—or hydroymethylcellulose, hydroxyethylstarch, dextrans, and polyvinylpyrrolidon (Kollidon). Specifically, by obtaining a slow release profile of the drug would reduce the inhalation frequency to once or twice daily.

It may also be advantageous to employ surfactants in the formulations of the invention. Surfactants or anti-adsorbents that prove useful include polyoxyethylenesorbitans, polyoxyethylenesorbitan monolaurate, polysorbate-20, such as Tween-20™, polysorbate-80, and genapol, vitamin E-TPGS and lecithins or lecithin constituents. For a potential reduction of drug adhesion or adsorption and solubilization of combination drugs and better lubrication, the composition may optionally further contain surfactants regarded as generally regarded as safe (GRAS) for inhalation, such as polysorbates, vitamin-TPGS and lecithins.

Without further elaboration, it is believed that one skilled in the art, using the preceding description, can utilize the invention to the fullest extent. The following examples for the inventive aqueous drug formulations for nebulization are illustrative only, and not limiting of the disclosure in any way whatsoever.

The examples described in the detailed description are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in the pharmacology, medical or related fields are intended to be within the scope of the appended claims.

The disclosures of all references and cited publications are expressly incorporated by reference in their entireties to the same extent as if each were incorporated by reference individually.

What is claimed is:

1. An aerosol therapy kit, said kit comprising:
a nebulizer;
at least one vial containing an effective amount of at least one drug; and
at least one pre-printed prescription.

2. The therapy kit of claim 1, wherein the drug is a compound selected from the group consisting of anti-inflammatory compounds, anti-allergies, glucocorticoids, anti-infective agents, antibiotics, antifungals, antivirals, mucolytics, antiseptics, vasoconstrictors, wound healing agents, local anesthetics, peptides, and proteins.

3. The therapy kit of claim 1, wherein said nebulizer is appropriate for use with an adult.

4. The therapy kit of claim 1, wherein said nebulizer is appropriate for use with a child.

5. The therapy kit of claim 1, wherein said nebulizer is appropriate for use with an infant.

6. The therapy kit of claim 1, wherein the nebulizer is a jet nebulizer.

7. The therapy kit of claim 1, wherein in the nebulizer is a mesh nebulizer.

8. The therapy kit of claim 1, wherein the effective amount of the drug is used to treat a patient afflicted with a condition selected from the group consisting of asthma, acute lower respiratory tract infection, chronic obstructive pulmonary disease, acute bronchitis, bronchiectasis, environmental pulmonary diseases, pulmonary hypertension, mediastinal and pleural disorders, pneumonia, infant respiratory distress syndrome, croup, bronchitis, and pertussis.

9. The therapy kit of claim 1, further comprising:
nebulizer tubing; and
a mask.
